(19) United States
(12) Patent Application Publication
(10) Pub. No.: US 2022/0145409 A1
(43) Pub. Date: May 12, 2022

(54) Amelioration Based on Detection of Biological Cells or Biological Substances

(71) Applicant: Nano Global Corp., Austin, TX (US)

(72) Inventors: Steven Papermaster, Austin, TX (US); Zoltan Papp, Austin, TX (US); Christine Scheve, Austin, TX (US); Aaron Papermaster, Austin, TX (US); Eric Crawford, Austin, TX (US)

(21) Appl. No.: 17/434,550

(22) PCT Filed: Jan. 27, 2020
(86) PCT No.: PCT/US2020/015228

§ 371 Date: Aug. 27, 2021

Related U.S. Application Data
(60) Provisional application No. 62/797,703, filed on Jan. 28, 2019.

(51) Int. Cl. C12Q 3/00; C12Q 1/04

(52) U.S. Cl. CPC C12Q 3/00; C12Q 1/04

(57) ABSTRACT

Systems, methods, storage media, and computing platforms that facilitate amelioration based on detection of biological cells or biological substances are disclosed. Example implementations may: monitor a scene proximate to a monitoring device; detect a type or class of biological cells and/or substance amongst the in-scene biological cells and/or substances found in a scene proximate to a monitoring device; and respond to the detection in a manner that ameliorates the harmful effects of the detected type or class of biological cells and/or substances.

### BACKGROUND

In biology, a cell is the basic structural, functional, and biological unit of all known living organisms. A cell is the smallest unit of life that can replicate independently, and cells are often called the “building blocks of life.” The term “cell” itself is very common. Herein, the term “biological cell” is used to distinguish the term from other common uses in other fields.

Typically, biological cells consist of cytoplasm enclosed within a membrane, which contains many biomolecules such as proteins and nucleic acids. Organisms can be classified as single-celled or unicellular (consisting of a single cell; including bacteria) or multicellular (such as plants and animals). While the multicellular plants and animals are often visible to the unaided human eye, their individual cells are visible only under a microscope, with dimensions between 1 and 100 micrometers.

Common examples of biological cells are microbes. Microbes (i.e., microscopic organisms) are microscopic living things that are found nearly everywhere on our planet. Indeed, macroscopic living things (e.g., humans) normally co-exist peacefully with microbes. Indeed, many microbes are helpful or essential to a healthy life and a healthy ecosystem.

Unfortunately, some microbes are harmful microorganisms. These are typically called pathogens. A bacterium is an example of a pathogen that may infect a human and produce disease. For example, listeria produces listeriosis and staphylococcus produces a staph infection.

Modern hygiene and sanitation techniques and technology have greatly reduced the chances of encountering pathogens. However, they have not eliminated the risk. Indeed, many people still fall ill or worse by coming in contact with pathogens. Often, these pathogens are transferred from one surface (e.g., countertop) to another (e.g., a hand) by surface contact.

Since microbes, by their nature, are too small to be seen by the unaided eye, a person is unaware of the relative cleanliness of a surface before touching that surface. Since all typical surfaces have some microbes thereon, a person is typically unable to know how much or what kind of microbes are on a surface.

### SUMMARY

One aspect of the present disclosure relates to a system configured to facilitate amelioration based on detection of biological cells or biological substances. The system may include one or more hardware processors configured by machine-readable instructions. The processor(s) may be configured to monitor a scene proximate to a monitoring device. The scene may include biological cells and/or substances. The processor(s) may be configured to detect a type or class of biological cells and/or substance amongst the in-scene biological cells and/or substances found in a scene proximate to a monitoring device. The processor(s) may be configured to respond to the detection in a manner that ameliorates the harmful effects of the detected type or class of biological cells and/or substances.

Another aspect of the present disclosure relates to a method to facilitate amelioration based on detection of biological cells or biological substances. The method may include monitoring a scene proximate to a monitoring device. The scene may include biological cells and/or substances. The method may include detecting a type or class of biological cells and/or substance amongst the in-scene biological cells and/or substances found in a scene proximate to a monitoring device. The method may include responding to the detection in a manner that ameliorates the harmful effects of the detected type or class of biological cells and/or substances.

Yet another aspect of the present disclosure relates to a non-transient computer-readable storage medium having instructions embodied thereon, the instructions being executable by one or more processors to perform a method to facilitate amelioration based on detection of biological cells or biological substances. The method may include monitoring a scene proximate to a monitoring device. The scene may include biological cells and/or substances. The method may include detecting a type or class of biological cells and/or substance amongst the in-scene biological cells and/or substances found in a scene proximate to a monitoring device. The method may include responding to the detection in a manner that ameliorates the harmful effects of the detected type or class of biological cells and/or substances.

Still another aspect of the present disclosure relates to a computing platform configured to facilitate amelioration based on detection of biological cells or biological substances. The computing platform may include a non-transient computer-readable storage medium having executable instructions embodied thereon. The computing platform may include one or more hardware processors configured to execute the instructions. The processor(s) may execute the instructions to monitor a scene proximate to a monitoring device. The scene may include biological cells and/or substances. The processor(s) may execute the instructions to detect a type or class of biological cells and/or substance amongst the in-scene biological cells and/or substances found in a scene proximate to a monitoring device. The processor(s) may execute the instructions to respond to the detection in a manner that ameliorates the harmful effects of the detected type or class of biological cells and/or substances.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates conventional techniques to detect and identify pathogens.

FIG. 2 illustrates an example system in accordance with the present disclosure.

FIG. 3 illustrates an example system in accordance with the present disclosure.

FIG. 4 illustrates a system configured to facilitate amelioration based on detection of biological cells or biological substances, in accordance with one or more implementations.

FIGS. 5A and 5B illustrates a method (or portions thereof) to facilitate amelioration based on detection of biological cells or biological substances, in accordance with one or more implementations.

The Detailed Description references the accompanying figures. In the figures, the left-most digit(s) of a reference number identifies the figure in which the reference number first appears. The same numbers are used throughout the drawings to reference like features and components.

### DETAILED DESCRIPTION

Systems, methods, storage media, and computing platforms to facilitate amelioration based on detection of biological cells or biological substances are disclosed. Example implementations may: monitor a scene proximate to a monitoring device; detect a type or class of biological cells and/or substance amongst the in-scene biological cells and/or substances found in a scene proximate to a monitoring device; and respond to the detection in a manner that ameliorates the harmful effects of the detected type or class of biological cells and/or substances.

Based on an analysis of the data collected about the scene, it may be determined that pathobiological cells and/or substances exist. In some implementations, the cells may be detected or identified on or inside a human body. In some instances, this detection does not rely on later and further testing at another location, thus the detection is in-the-field.

Responding to the detection and identification of pathogens (i.e., disease-causing) and pathological (i.e., diseased) cells is an application of the technology described herein.

In some implementations of the technology described herein, disease-causing cells (e.g., pathogens) or disease-causing substances (e.g., toxins) may be detected or identified in situ or “in the lab.” As used herein, the term “in the lab” refers to the opposite of in situ. While the action may literally occur in a lab, the term more broadly refers to these actions (e.g., detection and identification) occurring not in place, not in the field, away from the location where the cells or substances are discovered, or with interference, contact, or adulteration by the tester or testing equipment. For example, growing collected cells in a petri dish occurs in the lab.

### Example Scenario

Listeria monocytogenes is a pathogen that causes listeriosis, which is an infection with symptoms of fever, vomiting, and diarrhea. This pathogen is an example of a pathobiological cell. Listeria can spread to other parts of the body and lead to more serious complications, like meningitis. Listeria is often transmitted by ready-to-eat foods, such as milk, cheese, vegetables, raw and smoked fish, meat, ice cream, and cold cuts. This early and rapid detection is desirable so that cross-contamination can be avoided and any problems immediately addressed.

These ready-to-eat foods are often mass produced in food factories. In these factories, there is little to no time to stop production to test to determine if a harmful pathogen (like listeria) exists on the food-production surfaces. Depending on the comprehensiveness and desired accuracy of the test, conventional techniques to detect the bacteria take as long as a week to as short as hours. Regardless of the particulars of the test, these conventional tests involve the manual collection of samples from various surfaces, cataloging these samples, and performing invasive testing (e.g., culturing, chemical reaction, antibodies, etc.).

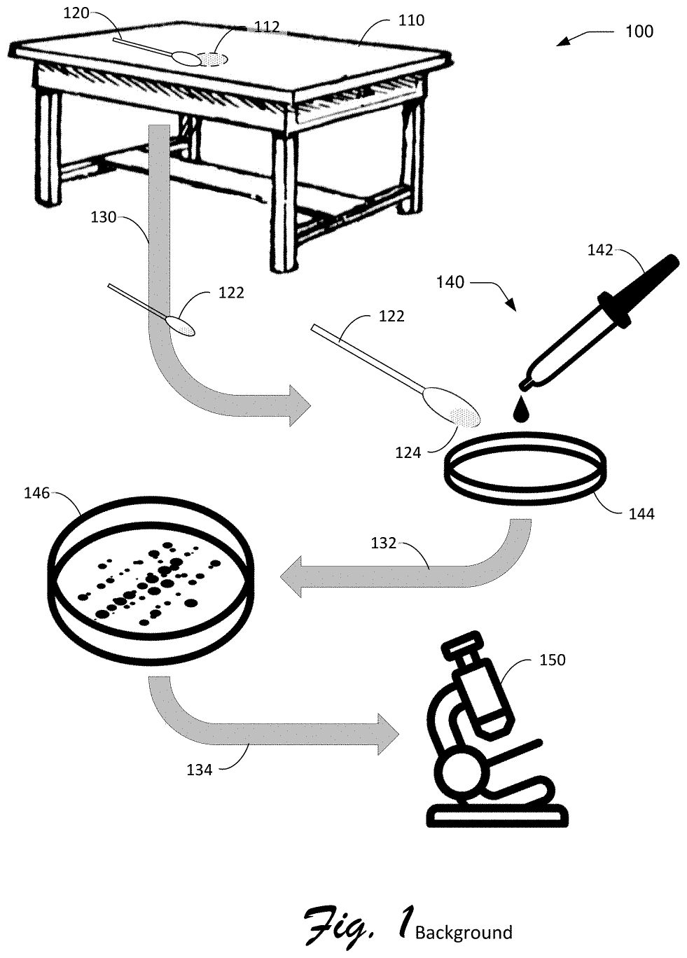

FIG. 1 illustrates conventional techniques 100 to detect and identify pathogens. Table 110 has a surface that, of course, has microbes thereon. This table 110 represents anything with a surface area that might have microbes living on it. For this discussion, assume that table 110 is in a commercial kitchen of a ready-to-eat food manufacturer. This manufacturer is concerned about Listeria in this kitchen. To detect the existence of Listeria in its kitchen, the manufacturer orders spot tests be performed in the kitchens.

To that end, a spot 112 on table 110 is selected for sampling. Using a sample-collection swab 120, a tester swipes the spot 112. Following arrow 130, a sample-collected swab 122 is carefully transported to a testing station 140 so as to avoid collateral and inadvertent collection of samples from other sources.

Typically, this testing station 140 is physically separated and distant from the sample spot 112 of the commercial kitchen where the sample was collected. The testing station 140 is often in a laboratory of a testing facility. With traditional methods, the sample microbes 124 of the sample-collected swab 122 are transferred to Petri dishes 144 for cultivation. At some point, chemicals 142 may be added to the cultured microbes of the Petri dishes 144 for various reasons, such as dyes to make them more visible.

Following arrows 132 and 134 and perhaps weeks or months, a petri dish 146 with the adultered (e.g., dyed) cultured microbes is ready to be examined under a microscope 150. Typically, a human examines the cultures under the microscope 150 and identifies pathogens amongst the cultured microbes based on many factors, but mostly the human's professional experience in identifying such microbes.

Traditional methods of testing like that demonstrated in FIG. 1, where sample microbes are cultivated in labs, are flawed. ‘Stressed’ cells will not grow in cultures (and will, therefore, produce negative results) despite the bacteria being present, live and potentially harmful. Also, this is the slowest form of testing.

Alternative conventional techniques, based on molecular/chemical methods, detect all cell types, but don't differentiate between live and harmless dead cells, which can remain after disinfection. Thus, these molecular/chemical methods may indicate a false positive for the presence of a pathogen when only dead cells of the pathogen are present.

Still, other conventional techniques use antibodies to test biofilms, which are groups of microbes where cells stick together on a surface. This technique requires the biofilms to be removed from the surface, treated with a particular antibody, and then tested to see if the biofilm fluoresces. This type of technique only tests for the particular pathogen that the introduced antibody interacts with.

Regardless of the conventional approach taken to detect pathogens or such dangers, the conventional response to correct the problem is a wholesale manually cleaning and sanitizing by a crew of humans with manual cleaning supplies. Once someone is sick, presenting symptoms, and manually diagnosed, then a doctor often proscribes antibiotics or antivirals to treat the sickness.

Example Electronic Device

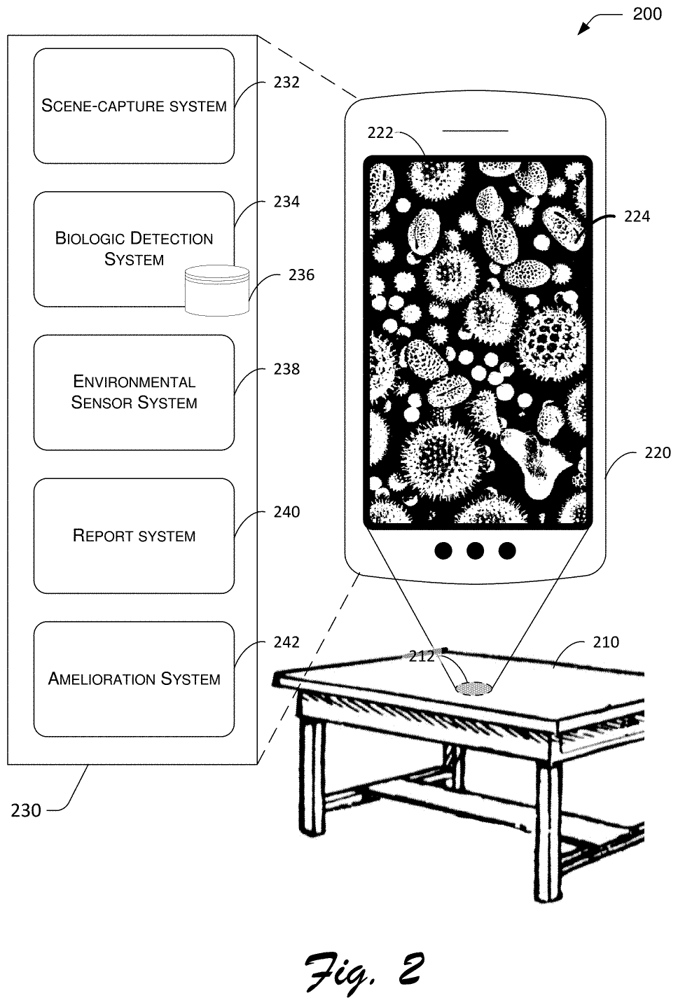

FIG. 2 illustrates an example scenario 200 in accordance with the technology described herein. The example scenario 200 includes a table 210. That table has a scene 212, which is an area of a surface in view of a camera (not shown) of a smartphone 220. Indeed, the camera captures an image 222 of scene 212. Just like reality, the scene 212 includes multiple microbes, but these microbes are not visible in scene 212.

For the example scenario 200, the microbes are described as in situ (i.e., in place) because they are examined, tested, etc. where they naturally live, inhabit, or exist. That is, the microbes are not in the lab. Herein, “in the lab” indicates that the microbes have been moved, relocated, or expatriated in order to perform the examination, testing, or the like. Other implementations of the technology described herein may involve microbes that are in the lab.

That image 222 is depicted as being viewed on a display of the smartphone 220. The image 222 has been sufficiently magnified to be able to see various in situ microbes of scene 212. And while not yet detected, one of these microbes is a pathogen 224.

The smartphone 220 is one example of an electronic device 230 in accordance with the technologies described herein. However, in other example scenarios, the electronic device 230 may be, for example, a tablet computer, a smartdevice, a standalone device, a collection of cooperative devices, a button-sized device, a device on a chip, an accessory to a smartphone or smartdevice, an ambulatory device, a robot, swallowable device, an injectable device, embedded within medical lab equipment, or the like.

As depicted, the electronic device 230 includes a scene-capture system, a biologic detection system (and object detection system) 234, a database 236, an environmental sensor system 238, a report system 240, and an amelioration system 242. These systems of the electronic device 230 are constructed from hardware, firmware, special-purpose components (e.g., sensors), and/or some combination thereof. These systems may, in some instances, include software modules as well.

The scene-capture system 232 is designed to obtain an image (e.g., image 222) of a scene (e.g., scene 212) that includes in situ biological cells therein. That is, there are biological cells located in a place (i.e., in-the-field) in the scene that is being captured by the scene-capture system. In some implementations, the scene-capture system 232 includes a camera to capture the visible part of the electromagnetic spectrum that is emitting or reflecting from the matter contained in the scene. In some implementations, the scene-capture system 232 includes components designed to capture non-visible parts of the electromagnetic spectrum (e.g., x-rays, infrared, gamma rays, ultraviolet, etc.) that is emitting or reflecting from the matter contained in the scene.

Examples of the action of obtaining (as performed by the scene-capture system 232) include measuring, collecting, accessing, capturing, procuring, acquiring, and observing. For example, the scene-capture system 232 may obtain the image by capturing the image using the charge-coupled device (CCD) of the digital camera. In another example, the scene-capturing system 232 may obtain the image by measuring the electromagnetic spectrum of the scene.

The obtained image is micrographic, spectrographic, digital, or some combination thereof. The obtained image is micrographic because it captures the elements in the scene that are on a microscopic scale. The obtained image is spectrographic because it captures elements in the scene by using equipment sensitive to portions of the electromagnetic spectrum (visible and/or non-visible portions). The obtained image is digital because it formats and stores the captured information as data capable of being stored in a machine, computer, digital electronic device, or the like.

While the thing that is captured is called an image, this image is not necessarily displayable as a two-dimensional depiction on a display screen (as shown in image 222). Rather, the image an array of data that represents the quantitative and qualitative nature of the electromagnetic spectrum (or some portion thereof) received by the components of the scene-capture system 232 when it was exposed to the scene (e.g., scene 212).

The biologic detection system 234 is designed to analyze the obtained image and detect the presence of one or more pathobiological cells amongst the in situ biological cells of the captured scene. In some implementations, the biologic detection system 234 may actually identify one or more particular cells and/or substances in the scene. In that case, it may be called a biologic identification system. Such a system may identify the particular pathobiological cells amongst the in situ biological cells. Thus, depending on the implementation, this system 234 may be referred to as biological-cell detection system or the pathobiological detection system.

To accomplish detection, the biologic detection system 234 may employ on and/or employ a database 236. This database 236 may be a database of pathobiologic-cellular signatures or training corpus. The biologic detection system 234 is a particular example of a biologic-cellular detection system. A training corpus is a database of numerous application-specific samples from which an AI/ML/DL engine “learns” and improves its capabilities and accuracy.

The biologic detection system 234 employs an AI/ML/DL engine to perform or assist in the performance of the detection and/or identification of one or more pathobiological cells. AI/ML/DL is short for artificial intelligence/machine learning/deep learning technology. Particular implementations may employ just an AI engine, just an ML engine, just a DL engine, or some combination thereof.

The AI/ML/DL engine may be implemented just on the smartphone 220 itself. In that case, the smartphone 220 need not communicate in real time with the platform (e.g., a remote computing system). In another implementation, the AI/ML/DL engine may be implemented just on the platform (thus remotely). In that case, the smartphone 220 communicates in real time (or nearly so) with the platform (e.g., a remote computing system). In still other implementations, the AI/ML/DL engine is implemented partially in both the smartphone 220 and the platform. In this way, the intensive processing is offloaded to the platform.

Some implementations of the biologic detection system 234 may perform its data analysis solely on the device without assistance from other devices, servers, or the cloud. Other implementations of the biologic detection system 234 may farm out all or nearly all of the data analysis to other devices, servers, or the cloud. In still other implementations, the data analysis may be shared amongst multiple devices and locations.

On its own or working with other devices or computer systems, the biologic detection system 234 analyzes the image of the scene to detect, determine, and/or identify the type or class of biological cells therein. It may do this, at least in part, by using distinguishing molecules of the cells that are observable using the electromagnetic spectrum. Is some implementations, other data (such as chemical reactions or excitation) may be included in the analysis.

Some of these molecules are indicative of certain classes, types, or particular cells. Such molecules are called marker biomolecules herein. The electronic device can determine which cell types or class are present in a captured scene by the on the particular ones of, the types of, and the proportions of the biomolecules detected therein. This may be accomplished, at least in part, by calculating probabilities of objects detected in the image.

The environmental sensor system 238 is designed to measure one or more environmental factors associated with the in situ biological cells or the environment surrounding the in situ biological cells. In some instances, the environmental sensor system 238 may be simply described as a sensor.

The report system 240 is designed to report detection and/or identification of the one or more pathobiological cells in the obtained image and in some implementations, the report system 240 is designed to associate the measured environmental factor with the obtained image and/or with the detected pathobiological cell.

The amelioration system 242 is designed to respond to the detection and/or identification in a manner that ameliorates the pathobiological nature of the detected/identified pathobiological cells.

The electronic device 230 may have a communications system to send or receive data from other similar electronic devices or centralized/distributed servers. The electronic device 230 may have enhanced processor or co-processor to perform image-capture and processing functions.

Of course, the example scenario 200 described above is one implementation that detects pathobiological cells. Other implementations of the technology described herein may detect or identify pathobiological substances rather than cells.

One or more of the systems of electronic device 230 may be characterized as a non-transitory computer-readable storage medium comprising instructions that when executed cause one or more processors of a computing device to perform the operations of that electronic device.

Environmental Factors

As indicated above, sensors obtain environmental factors related to, about, or near the scenes being observed. These may be called ambient factors. The sensors may measure or sense to obtain the environmental factors. In other instances, the factors may be accessed, acquired, procured, etc. via another source, sensor, memory, machine, etc.

The environmental factors are abiotic or biotic. However, there are other datapoints that may be gathered, but which are not expressly related to the environment. These may be called associated or observation-related factors.

An abiotic environmental factor is associated with non-biological sources. That is, the source of the thing being measured is not related to a living or recently living thing.

Examples of abiotic environmental factor include ambient temperature, timestamp (e.g., time and date), moisture, humidity, radiation, the amount of sunlight, and pH of a water medium (e.g., soil) where a biological cell lives. Other examples of abiotic environmental factors include barometric pressure; ambient sound; indoor location; ambient electromagnetic activity; velocity; acceleration; inertia; ambient lighting conditions; WiFi fingerprint; signal fingerprints; GPS location; geolocation; airborne particle counter; chemical detection; gases; radiation; air quality; airborne particulate matter (e.g., dust, 2.5 PPM, 10 PPM, etc.); atmospheric pressure; altitude; Geiger counter; proximity detection; magnetic sensor; rain gauge; seismometer; airflow; motion detection; ionization detection; gravity measurement; photoelectric sensor; piezo capacitive sensor; capacitance sensor; tilt sensor; angular momentum sensor; water-level (i.e., flood) detection; flame detector; smoke detector; force gauge; ambient electromagnetic sources; RFID detection; barcode reading; or some combination thereof.

A biotic environmental factor is one having a biologic source. Example of such include the availability of food organisms and the presence of conspecifics, competitors, predators, and parasites.

While it is not an environment factor, per se, the observation-related or associated factor is described here. The associated or observation-related factor may be a measurement of quality, quantity, and/or characteristic of the environment about the observation itself or related to the environment from which the subject is observed or was obtained. They may also be data that a human or computer associated with other environmental factors or the scene.

Examples of the observation-related or associated factor include nearby traffic patterns or noises; tracking the movements of particular individuals (e.g., via employee badges or security cameras); visitors; patients, budgets of related departments; and the like.

Herein, a known sensor or measurement device may be listed as an example of an environmental factor. For example, Geiger counter and seismometer are listed as examples. It should be understood that the relevant factor for least listed examples is the measurements typically made by such devices. Thus, the obtained factor for example Geiger counter is radiation and the obtained factor for the example seismometer is the motion of the ground.

Network of Monitoring Devices

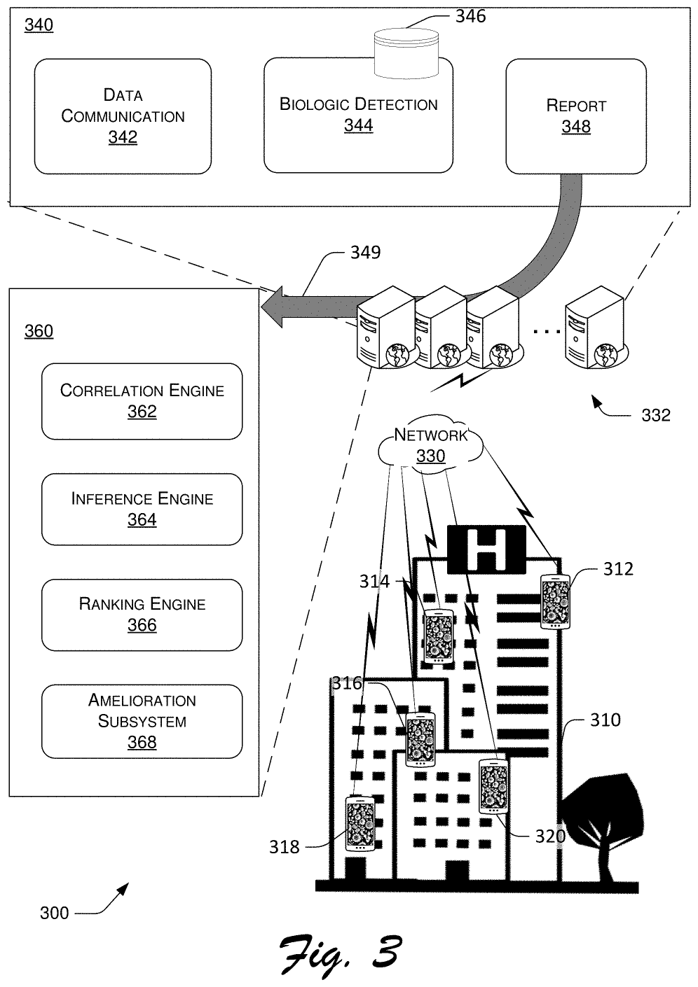

FIG. 3 is an illustration of an infrastructure 300 that facilitates data collection and data analysis of the technology described herein. The data collection, for example, may occur across a network of various widths (i.e., horizontally) and depths (i.e., vertically). Consider for this example a single building. That building is called the Hope Hospital 310.

The Hope Hospital 310 has many different floors, rooms, departments, etc. For illustration purpose, the Hope Hospital 310 has four floors of five rooms each. And each floor is a department.

The Hope Hospital 310 has installed a variety of electronic monitoring devices. These devices are like smartphone 220 of example scenario 200. However, these electronic monitoring devices may be stationary and special-purpose. These electronic monitoring devices are called NANOBOT™ devices herein. However, these NANOBOT™ devices are special-purpose devices rather than a smartphone. That is, the NANOBOT™ devices are designed and built for the specialized and particular purpose of the functionality described herein.

In this example, there are multiple NANOBOT™ devices placed throughout the hospital. Indeed, there could be multiple devices in each room. For example, a NANOBOT™ device may be installed on the bed, the bathroom counter, hand sanitizer dispenser, the faucet handle, the air vent, and the like. In addition, other NANOBOT™ devices may be installed in the ductwork of the HVAC.

As depicted, stationary device 312 is mounted on the wall in a room of patient X, device 314 is on the ceiling of a room of patient Y, device 316 is on the bed of patient Z, device 318 is mounted is in the ducting of the third floor, and device 320 is a portable device carried by a nurse.

Each of these NANOBOT™ devices are installed to monitor biological cells and/or substances and environmental factors in their proximity. The devices will detect and/or identify the particular cells and/or substances in their proximity. In addition, the ongoing monitoring of these devices enable the tracking of changes in the detected and/or identified microscopic lifeforms, for example, in their proximity.

In addition, each of these NANOBOT™ devices include a sensor or sensors for monitoring one or more environmental factors, such as ambient temperature, humidity, indoor location, and the like. Each device tracks its proximate factors and microscopic lifeforms over time.

In addition to the stationary NANOBOT™ devices, other electronic devices may be used in the Hope Hospital 310. For example, there may be mobile or ambulatory devices that are specially designed to do the same functions. These devices may be affixed to a mobile or portable platform. Alternatively, these devices may have the ability to relocate on their own power or volition.

For example, NANOBOT™ device may be affixed to a cart, robotic medication dispenser, a chart, and the like. As such, the NANOBOT™ device tracks the environmental factors and biological cells proximate the device as the thing to which it is affixed is moved throughout the hospital. As such, the indoor location of the device changes as well.

Similarly, a NANOBOT™ device may have its own mechanism for self-propulsion. It may have electric motors, wheels, and self-navigational capability to travel the hallways, walls, and ducts of the building. In addition, some self-propelled NANOBOT™ devices may travel in the air, for example, like a so-called drone (i.e., unmanned aerial vehicle). In some instances, a self-propelled NANOBOT™ device may travel on and/or within liquid.

The self-propelled NANOBOT™ device may wander around the hospital or other space to generate a comprehensive amount of monitoring data for such space or a portion thereof. Alternatively, the self-propelled NANOBOT™ device may travel a pre-determined path or navigate its own path. In doing so, the device is tracking data as it travels and/or at particular points along the path.

In addition, humans may carry smartphone or some form of smartphone accessory (e.g., watch) that is capable of performing these functionalities. Indeed, this type of mobile device may perform these functions actively and/or passively. That is, the user may actively choose when and where to perform measurements and/or the device may choose when and where to perform measurements.

These various devices in the Hope Hospital 310 may be interconnected with each other and/or connected to a common or interconnected network 330. For example, the stationary NANOBOT™ devices may be connected to each other view a peer-to-peer mesh wireless network. Devices may be connected via a wireless access point (WAP) or via short-range wireless signal (e.g., BLUETOOTH). In turn, these networks may be connected to other networks (such as the so-called Internet) and to centralized or distributed computing center. For example, all of the stationary NANOBOT™ devices in a room may be connected at a single nearby WAP that is connected to the so-called cloud where the acquired data is stored in a cloud-based storage system. The network 330 represents any and all of these suitable communication networks.

Data Collection

Each device in the Hope Hospital 310 is configured to monitor its proximate area. The devices monitor for biological cells and various environmental factors, such as location, temperature, and humidity. Each device may be configured to collect data in a manner like that smartphone 220 of example scenario 200.

Once the data is collected by a monitoring device, it may be locally analyzed or the raw data may be transferred or uploaded to a centralized and/or distributed computing system. Supersystem 332 is depicted as a several servers. This represents the centralized and/or distributed computing system.

If analyzed locally, the collected data may be fully or partially analyzed locally. With local analysis, some or all of the processing that is necessary to detect and/or identify a class or type of biological cell is performed on the monitoring electronic device. Indeed, if fully processed locally, the monitoring device may form a conclusion regarding the type/class of cell in a monitored scene.

In some instances, the raw monitor data or partially processed data may be uploaded or transferred to a computing system. For example, each floor of the Hope Hospital 310 may have its own dedicated computer to store monitor data of the devices of that floor. That floor computer may perform some or all of the calculations needed to determine that type/class of the cells in the monitored scenes. Alternatively or in addition, each building may have its own computer; each campus has its own computer; each city has its own computer; each region has its own computer; etc. Alternatively or in addition, all of this data is transferred to the “cloud” for storage and processing overall or at each level.

In addition, individuals may collect data for their own personal reasons. For example, a visitor may collect data in the cafeteria of the hospital so that she knows how clean the surfaces are. This data and/or its analysis may be uploaded to the so-called cloud. That is, the data collection may be crowdsourced or available to the individual alone.

This data may be collected in a coordinated fashion by the Hope Hospital 310 or an agency working on their behalf. The collection and analysis of this data may be performed by the Hope Hospital 310. In addition, the collection and analysis of the data of Hope Hospital 310 may be a service that the Hope Hospital 310 subscribes to.

Furthermore, a service may collect data from many and various locations in an anonymous fashion to protect the private data of each identifiable customer. With the data collected from many different locations and customers, the service may analyze the data to find meta-trends and meta-correlations.

The supersystem 332 includes one or both systems 340 and 360. System 340 is primarily for the collection and analysis of image-based data. System 360 is primarily for the inferences or conclusions of the collected and analyzed data. The supersystem may be called the “platform” herein.

System 340 includes a data communications subsystem 342, a biologic detection subsystem 344, and a report subsystem 348.

The data communication subsystem 342 obtains (e.g., via wireless communication) image-based data from one or more of multiple remote monitoring devices. The image-based data from each monitoring device is based on (e.g., derived from) one or more images of a scene proximate to that monitoring device. The proximate scene includes biological cells and/or substances therein.

The data communication subsystem 342 also obtains (e.g., via wireless communication) environmental data from one or more of the multiple remote monitoring devices. The environmental data being based on an environmental factor associated with the in-scene biological cells and/or substances of each device or the environment surrounding the in-scene biological cells and/or substances of each device.

The biologic detection subsystem 344 analyzes the image-based data and/or the environmental data. Based on that analysis, the biologic detection subsystem 344 detects and/or identifies a type or class of biological cells and/or substance amongst the in-scene biological cells and/or substances of each device or amongst several of the devices.

To accomplish detection, the biologic detection subsystem 344 may employ on and/or employ a database 346. This database 346 may be a database of biologic-cellular or biologic-substantive signatures. This may be called a training corpus. A training corpus is a database of numerous application-specific samples from which the AI/ML/DL engine “learns” and improves its capabilities and accuracy.

The biologic detection subsystem 344 employs an AI/ML/DL engine to perform or assist in the performance of the detection and/or identification of one or more biological cells and/or substances.

The AI/ML/DL engine functionality may be split across the platform. That is, the devices may perform pre-processing of the image using AI/ML/DL engine and send the results as the image-based data to the system 340 for further processing herein. In that case, the device 314 communicates in real time (or nearly so) with the platform. In this way, the intensive processing is offloaded from the devices to the platform.

The biologic detection subsystem 344 analyzes the image-based data of the scene to detect, determine, and/or identify the type or class of biological cells and/or substances therein. It may do this, at least in part, by using distinguishing molecules of the cells that are observable using the electromagnetic spectrum. In some implementations, other data (such as chemical reactions or excitation) may be included in the analysis.

The report subsystem 348 reports the detection and/or identification of the type of biological cells and/or substances in the scene proximate to the monitoring device. The report subsystem 348 may send 349 its results and associated data (image-based data and/or environmental data) to the system 360.

Data Analysis

As discussed herein, an electronic device captures a scene that has biological cells therein. In some instances, these biological cells may be described as in situ (i.e., in place) because they are monitored, examined, tested, etc. where they naturally live, inhabit, or exist. That is, the biological cells have not been moved, relocated, or expatriated in order to perform the examination, testing, or the like.

Using a camera or digital or other imaging technology, the electronic device captures a portion of the electromagnetic spectrum that is emitting or reflecting from the matter contained in the scene. The obtained image is micrographic, spectrographic, digital, or some combination thereof. The obtained image is micrographic because it captures elements in the scene that are on a microscopic scale. The obtained image is spectrographic because it captures elements in the scene by using equipment sensitive to portions of the electromagnetic spectrum (visible and/or non-visible portions). The obtained image is digital because it formats and stores the captured information as data capable of being stored in a machine, computer, digital electronic device, or the like.

While the thing that is captured is called an image, this image is not necessarily displayable as a two-dimensional depiction on a display screen. Rather, the image is an array of data that represents the quantitative and qualitative nature of the electromagnetic spectrum (or some portion thereof) received by the camera of the electronic device when it was exposed to the scene.

On its own or working with other devices or computer systems, the electronic device analyzes the image of the scene to detect, determine, and/or identify the type or class of biological cells therein. It may do this, at least in part, by using distinguishing molecules of the cells that are observable using the electromagnetic spectrum. That is, the electronic device captures the observable electromagnetic spectrum (e.g., visible and/or non-visible) that is reflected, scattered, emitting, etc. from the in-situ cells of a captured scene to determine the molecules of those cells.

Some of these molecules are indicative of certain classes, types, or particular cells. Such molecules are called marker biomolecules herein. The electronic device can determine which cell types or class are present in a captured scene by the on the particular ones of, the types of, and the proportions of the biomolecules detected therein.

In addition, the electronic device may includes or may have one or more environmental sensors that are designed to measure one or more environmental factors associated with the in situ biological cells or the environment surrounding the in situ biological cells.

The electronic device may have or connect to a report system that is designed to report a detection and/or identification of the one or more cell types or classes in the obtained image and in some implementations, the report system is designed to associate the measured environmental factor(s) with the obtained image and/or with the detected cell.

Amelioration & Delivery

The system 360 includes an amelioration subsystem 368 that ameliorates (e.g., fixes) problems discovered by the reporting subsystem and/or the correlation or inference engines. Indeed, the amelioration subsystem 368 may trigger a delivery of the amelioration in response to such problems.

The amelioration subsystem 368 performs amelioration actions. Examples of such actions include (by way of example and not limitation):
introducing an active material (e.g., sanitizer, ultraviolet light, cleaning fluid/spray) to a physical location of the pathobiological cell and/or substance to neutralize (e.g., clean, reduce, kill, or eliminate) the pathobiological nature of the detected/identified pathobiological cell and/or substance;
formulating one or more therapeutics for one or more individuals associated with the scene, the formulated therapeutics being configured to treat one or more conditions associated with the detected type of biological cells and/or substances;
selecting one or more therapeutics for one or more individuals associated with the scene, the selected therapeutics being configured to treat one or more conditions associated with the detected type of biological cells and/or substances;
selecting one or more therapeutics for one or more individuals associated with the scene and a dosage of such therapeutics, the selected dosage of the therapeutics being configured to treat one or more conditions associated with the detected type of biological cells and/or substances;
introducing therapeutic to a physical location of the biological cell and/or substance to neutralize the biological nature of the detected type of biological cells and/or substances;
introducing therapeutics to one or more individuals associated with the scene, the therapeutics being configured to treat one or more conditions associated with the detected type of biological cells and/or substances (in some instances, the therapeutics is introduced to the one or more individuals before the one or more individuals have presented any or sufficient symptoms to otherwise warrant the therapeutics to treat one or more conditions associated with the detected type of biological cells and/or substances);
internal releasing preloaded therapeutic to one or more individuals associated with the scene by one or more implanted therapeutic delivery devices;
transdermally releasing preloaded therapeutic to one or more individuals associated with the scene by one or more transdermal therapeutic delivery devices;
topical application of a dosage of a therapeutic to one or more individuals associated with the scene by one or more dermal therapeutic delivery devices;
aeriated release of an airborne therapeutic in an area that includes or is proximate to the scene being monitored by one or more therapeutic delivery devices;
physical delivery of a package containing a therapeutic for one or more individuals associated with the scene;
dispatch or request a visit by a robot or human to a physical location of the pathobiological cell and/or substance to neutralize (e.g., clean, reduce, kill, or eliminate) the pathobiological nature of the detected/identified pathobiological cell and/or substance;
dispatch or request a visit by a robot or human to a physical location of the pathobiological cell and/or substance to document (e.g., photograph and measure) the conditions around the pathobiological cell and/or substance (e.g., a macroscopic photograph of the physical location);
activate an operation of a proximate electronic device or system that is proximate a physical location of the pathobiological cell and/or substance to neutralize (e.g., clean, reduce, kill, or eliminate) the pathobiological nature of the detected/identified pathobiological cell and/or substance;
activate an operation of a proximate camera that is proximate a physical location of the pathobiological cell and/or substance to document (e.g., photograph and video) the area around the pathobiological cell and/or substance;
trigger formulation of a customized therapeutic and ordering delivery of that therapeutic to specific patients, locations, organizations, and/or regions;
trigger selection of an available therapeutic and ordering delivery of that therapeutic to specific patients, locations, organizations, and/or regions; and/or
a combination thereof.

In some instances, the amelioration operation may include mitigation. For example, a recommendation may be made and/or directions given to institute treatment for an allergy if mold is present, increase the frequency of cancer check-ups, reallocate cleaning resources, etc.

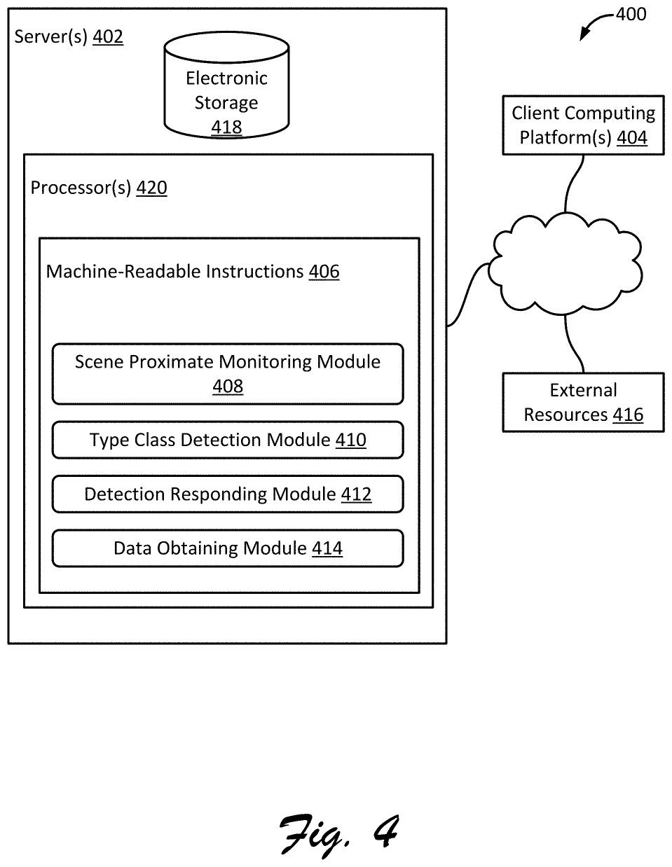

FIG. 4 illustrates a system 400 configured to facilitate amelioration based on detection of biological cells or biological substances, in accordance with one or more implementations. The system 400 is an example of the amelioration system 242 of electronic device 200 and the amelioration subsystem 368 of system 300.

In some implementations, system 400 may include one or more servers 402. Server(s) 402 may be configured to communicate with one or more client computing platforms 404 according to a client/server architecture and/or other architectures. Client computing platform(s) 404 may be configured to communicate with other client computing platforms via server(s) 402 and/or according to a peer-to-peer architecture and/or other architectures. Users may access system 400 via client computing platform(s) 404.

Server(s) 402 may be configured by machine-readable instructions 406. Machine-readable instructions 406 may include one or more instruction modules. The instruction modules may include computer program modules. The instruction modules may include one or more of scene proximate monitoring module 408, type class detection module 410, detection responding module 412, data obtaining module 414, and/or other instruction modules.

Scene proximate monitoring module 408 may be configured to monitor a scene proximate to a monitoring device. A scene is proximate a monitoring device when it is capable of being monitored by that device for the purposes of the device. For example, a scene is proximate a camera of the monitoring device when it is in a line of sight of the camera and close enough for the camera to capture sufficient details to make effective determinations of the contents of the scene. If, for example, the monitoring device is monitoring the sounds being emitted from the scene, then the scene would be proximate to a microphone (which would be the monitoring device in this example) when that microphone can pick up relevant sounds over the ambient noise.

The monitoring and detecting actions may be performed physically and temporally separate and at a distance from each other. The monitoring may include retaining samples of the in-scene biological cells and/or substances during a period of time for subsequent analysis. The monitoring may include actively monitoring a selection of biomarker indicators of health through transdermal collection. The scene may include biological cells and/or substances.

Type class detection module 410 may be configured to detect a type or class of biological cells and/or substance amongst the in-scene biological cells and/or substances found in a scene proximate to a monitoring device. The type or class of detected biological cell and/or substance may be a pathobiological cell and/or substance. By way of non-limiting example, the pathobiological biological cells may include pathologic cells, diseased cells, cancer cells, infectious agents, pathogens, bioagents, disease-producing agents, or combination thereof. By way of non-limiting example, the in-scene biological cells and/or substances may include biological cells and/or substances that are characterized as physically located on a surface, physically located in a medium, undisturbed in their environment, undisturbed and unadulterated, physically located on a surface in a manner that is undisturbed and unadulterated, not relocated for the purpose of image capture, unmanipulated for the purpose of image capture, or on a surface that is unaffected by the monitoring device. The detection may include operations to isolate a biological cell and/or substance using the image-based data of the scene proximate to the monitoring device.

The detection may include operations to provide the image-based data of the scene proximate to the monitoring device to a trained biological detection engine. The detection may include operations to receive a positive indication from the biological detection engine that the scene proximate to the monitoring device includes a biological cell and/or substance therein and/or identity of that biological cell and/or substance. The detection may include operations to provide the image-based data of the scene proximate to the monitoring device to a trained biological detection engine. The detection may include operations to receive a positive indication from the biological detection engine that the scene proximate to the monitoring device includes a biological cell and/or substance therein and/or identity of that biological cell and/or substance. The image-based data of the scene proximate to the monitoring device may include data captured from the visible and/or non-visible electromagnetic spectrum of the scene. The report system to report an identification of the biological cell and/or substance of the scene proximate to the monitoring device.

The report of the report system may indicate that the type of biological cells and/or substances of the scene proximate to the monitoring device is a category flagged for further research and inquiry. By way of non-limiting example, in whole or in part, the monitoring device may be selected from a group consisting of a system-on-a-chip, a device-on-a-chip, a smartdevice, a computer, an ambulatory device, a microscope, a mobile device, and a wireless device.

Detection responding module 412 may be configured to respond to the detection in a manner that ameliorates the harmful effects of the detected type or class of biological cells and/or substances. By way of non-limiting example, the biological cells may be characterized as cells of a multicell biological organism, cells of a tissue or organ of a multicell biological organism, cells of a tumor or growth multicell biological organism, single-celled organism, microbes, microscopic organisms, single-celled organism, living things that are too small to be seen with a human's naked eye, a biological creature that can only be seen by a human with mechanical magnification, microscopic spores, or a combination thereof. By way of non-limiting example, the biological cells may be characterized as microbes that are characterized as, single-celled organisms, bacteria, archaea, fungi, mold, protists, viruses, microscopic multi-celled organisms, algae, bioagents, spores, germs, prions, or a combination thereof.

Data obtaining module 414 may be configured to obtain environmental data based on an environmental factor associated with the in-scene biological cells and/or substances or the environment surrounding the in-scene biological cells and/or substances. By way of non-limiting example, the environmental factors on which the environmental data may be based includes biotic, abiotic, and associated factors.

In some implementations, the amelioration action may include introducing an active material to a physical location of the biological cell and/or substance to neutralize the biological nature of the detected type of biological cells and/or substances. In some implementations, the amelioration action may include formulating a therapeutics for one or more individuals associated with the scene. In some implementations, the formulated therapeutics may be configured to treat one or more conditions associated with the detected type of biological cells and/or substances. In some implementations, the amelioration action may include selecting one or more therapeutics for one or more individuals associated with the scene. In some implementations, the selected therapeutics may be configured to treat one or more conditions associated with the detected type of biological cells and/or substances.

In some implementations, the amelioration action may include selecting one or more therapeutics for one or more individuals associated with the scene and a dosage of such therapeutics. In some implementations, the selected dosage of the therapeutics may be configured to treat one or more conditions associated with the detected type of biological cells and/or substances. In some implementations, the amelioration action may include introducing therapeutic to a physical location of the biological cell and/or substance to neutralize the biological nature of the detected type of biological cells and/or substances. In some implementations, the amelioration action may include introducing therapeutics to one or more individuals associated with the scene. In some implementations, the therapeutics may be configured to treat one or more conditions associated with the detected type of biological cells and/or substances. In some implementations, the therapeutics may be introduced to the one or more individuals before the one or more individuals have presented any or sufficient symptoms to otherwise warrant the therapeutics to treat one or more conditions associated with the detected type of biological cells and/or substances.

In some implementations, the amelioration action may include dispatching or requesting a visit by a robot or human to a physical location of the biological cell and/or substance to neutralize the biological nature of the detected type of biological cells and/or substances. In some implementations, the amelioration action may include dispatching or requesting a visit by a robot or human to a physical location of the detected type of biological cells and/or substances to document the conditions around the detected type of biological cells and/or substances. In some implementations, the amelioration action may include activating an operation of a proximate electronic device or system that is proximate a physical location of the detected type of biological cells and/or substances to neutralize the biological nature of the detected type of biological cells and/or substances. In some implementations, the amelioration action may include activating an operation of a proximate camera that is proximate a physical location of the detected type of biological cells and/or substances to document the area around the detected type of biological cells and/or substances. In some implementations, the amelioration action may include internal releasing preloaded therapeutic to one or more individuals associated with the scene by one or more implanted therapeutic delivery devices. In some implementations, the amelioration action may include transdermally releasing preloaded therapeutic to one or more individuals associated with the scene by one or more transdermal therapeutic delivery devices.

In some implementations, the amelioration action may include topical application of a dosage of a therapeutic to fr one or more individuals associated with the scene by one or more dermal therapeutic delivery devices. In some implementations, the amelioration action may include aeriated release of an airborne therapeutic in an area that includes or is proximate to the scene being monitored by one or more therapeutic delivery devices. In some implementations, the amelioration action may include physical delivery of a package containing a therapeutic for one or more individuals associated with the scene. In some implementations, the in-scene biological cells and/or substances may be in situ. In some implementations, by way of non-limiting example, the in-scene biological cells may and/or substances include biological substances that are characterized as volatile organic compounds organic matter, chemical substances present or produced by living organisms, biomolecules, biogenic substances, biotic materials, biomass, body fluid, cellular components, tissue, viable materials, bio-based materials, biocomposites, biomaterials, biological materials, biologic particulate matter, or a combination thereof. In some implementations, the environmental factors may include those associated with conditions conducive to the growth and spread of pathogens through temperature and humidity sensors.

In some implementations, by way of non-limiting example, the environmental factors may be selected from a group consisting of temperature, timestamp, humidity, barometric pressure, ambient sound, location, ambient electromagnetic activity, ambient lighting conditions, wifi fingerprint, gps location, airborne particle counter, chemical detection, gases, radiation, air quality, airborne particulate matter, atmospheric pressure, altitude, geiger counter, proximity detection, magnetic sensor, rain gauge, seismometer, airflow, motion detection, ionization detection, gravity measurement, photoelectric sensor, piezo capacitive sensor, capacitance sensor, tilt sensor, angular momentum sensor, water-level detection, flame detector, smoke detector, force gauge, ambient electromagnetic sources, rfid detection, barcode reading, and a combination thereof. In some implementations, the detection may include operations to access a database of signatures of biological cells and/or substances. In some implementations, the detection may include operations to correlate the isolated biological cell and/or substance to at least one signature in the database. In some implementations, the detection may include operations to determine that the correlation identifys the isolated biological cell and/or substance as being a biological cell and/or substance. In some implementations, the detection may include operations to, in response to that correlation determination, label the isolated biological cell and/or substance as being the determined biological cell and/or substance. In some implementations, the trained biological detection engine may be an AI/ML/DL engine trained to detect and/or identify biological cells and/or substances based on a training corpus of signatures of biological cells and/or substances.

In some implementations, the trained biological detection engine may be an AI/ML/DL engine trained to detect and/or identify biological cells and/or substances based on a training corpus of signatures of biological cells and/or substances. In some implementations, by way of non-limiting example, the report of the report system may be characterized by performing operations that send a communication to a human or machine that is designated to receive such communications via wired or wireless communications mechanisms, send a notification to a human or machine that is designated to receive such notification via wired or wireless communications mechanisms, update a database designated to receive such updates via wired or wireless communications mechanisms, store the detection in a memory, or a combination thereof.

In some implementations, server(s) 402, client computing platform(s) 404, and/or external resources 416 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 402, client computing platform(s) 404, and/or external resources 416 may be operatively linked via some other communication media.

A given client computing platform 404 may include one or more processors configured to execute computer program modules. The computer program modules may be configured to enable an expert or user associated with the given client computing platform 404 to interface with system 400 and/or external resources 416, and/or provide other functionality attributed herein to client computing platform(s) 404. By way of non-limiting example, the given client computing platform 404 may include one or more of a desktop computer, a laptop computer, a handheld computer, a tablet computing platform, a NetBook, a Smartphone, a gaming console, and/or other computing platforms.

External resources 416 may include sources of information outside of system 400, external entities participating with system 400, and/or other resources. In some implementations, some or all of the functionality attributed herein to external resources 416 may be provided by resources included in system 400.

Server(s) 402 may include electronic storage 418, one or more processors 420, and/or other components. Server(s) 402 may include communication lines, or ports to enable the exchange of information with a network and/or other computing platforms. Illustration of server(s) 402 in FIG. 4 is not intended to be limiting. Server(s) 402 may include a plurality of hardware, software, and/or firmware components operating together to provide the functionality attributed herein to server(s) 402. For example, server(s) 402 may be implemented by a cloud of computing platforms operating together as server(s) 402.

Electronic storage 418 may comprise non-transitory storage media that electronically stores information. The electronic storage media of electronic storage 418 may include one or both of system storage that is provided integrally (i.e., substantially non-removable) with server(s) 402 and/or removable storage that is removably connectable to server(s) 402 via, for example, a port (e.g., a USB port, a firewire port, etc.) or a drive (e.g., a disk drive, etc.). Electronic storage 418 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 418 may include one or more virtual storage resources (e.g., cloud storage, a virtual private network, and/or other virtual storage resources). Electronic storage 418 may store software algorithms, information determined by processor(s) 420, information received from server(s) 402, information received from client computing platform(s) 404, and/or other information that enables server(s) 402 to function as described herein.

Processor(s) 420 may be configured to provide information processing capabilities in server(s) 402. As such, processor(s) 420 may include one or more of a digital processor, an analog processor, a digital circuit designed to process information, an analog circuit designed to process information, a state machine, and/or other mechanisms for electronically processing information. Although processor(s) 420 is shown in FIG. 4 as a single entity, this is for illustrative purposes only. In some implementations, processor(s) 420 may include a plurality of processing units. These processing units may be physically located within the same device, or processor(s) 420 may represent processing functionality of a plurality of devices operating in coordination. Processor(s) 420 may be configured to execute modules 408, 410, 412, and/or 414, and/or other modules. Processor(s) 420 may be configured to execute modules 408, 410, 412, and/or 414, and/or other modules by software; hardware; firmware; some combination of software, hardware, and/or firmware; and/or other mechanisms for configuring processing capabilities on processor(s) 420. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 408, 410, 412, and/or 414 are illustrated in FIG. 4 as being implemented within a single processing unit, in implementations in which processor(s) 420 includes multiple processing units, one or more of modules 408, 410, 412, and/or 414 may be implemented remotely from the other modules. The description of the functionality provided by the different modules 408, 410, 412, and/or 414 described below is for illustrative purposes, and is not intended to be limiting, as any of modules 408, 410, 412, and/or 414 may provide more or less functionality than is described. For example, one or more of modules 408, 410, 412, and/or 414 may be eliminated, and some or all of its functionality may be provided by other ones of modules 408, 410, 412, and/or 414. As another example, processor(s) 420 may be configured to execute one or more additional modules that may perform some or all of the functionality attributed below to one of modules 408, 410, 412, and/or 414.

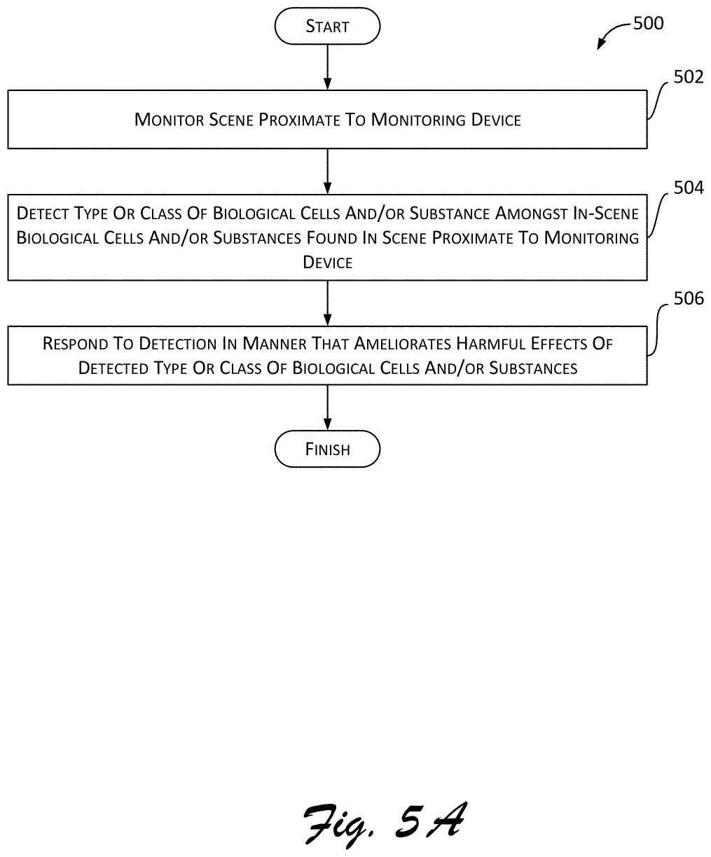

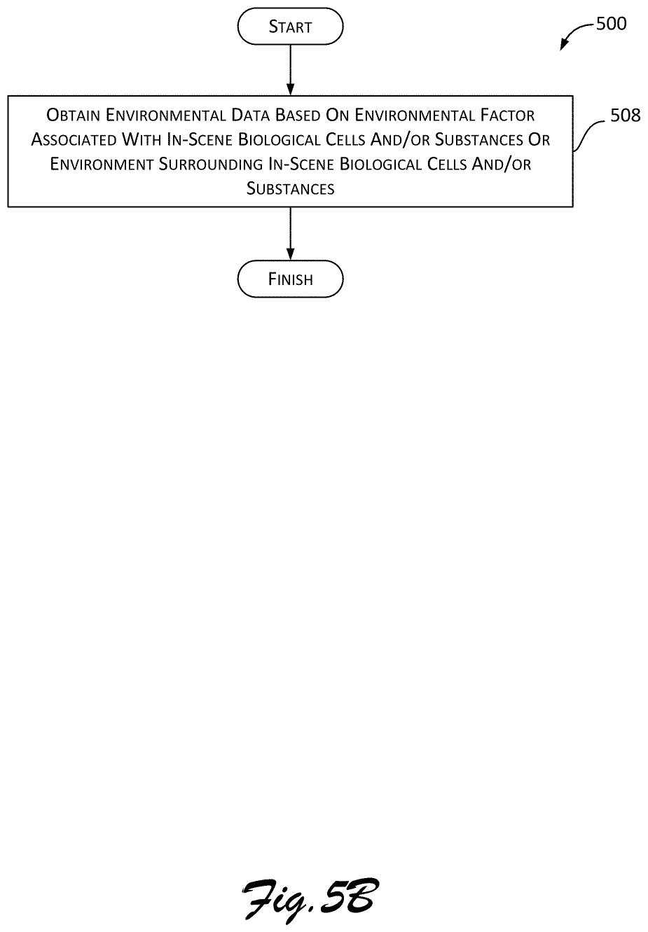

FIGS. 5A and/or 5B illustrates a method 500 that facilitates amelioration based on detection of biological cells or biological substances, in accordance with one or more implementations. The operations of method 500 presented below are intended to be illustrative. In some implementations, method 500 may be accomplished with one or more additional operations not described, and/or without one or more of the operations discussed. Additionally, the order in which the operations of method 500 are illustrated in FIGS. 5A and/or 5B and described below is not intended to be limiting.

In some implementations, method 500 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

FIG. 5A illustrates method 500, in accordance with one or more implementations.

An operation 502 may include monitoring a scene proximate to a monitoring device. The scene may include biological cells and/or substances. Operation 502 may be performed by one or more hardware processors configured by machine-readable instructions including a module that is the same as or similar to scene proximate monitoring module 408, in accordance with one or more implementations.

An operation 504 may include detecting a type or class of biological cells and/or substance amongst the in-scene biological cells and/or substances found in a scene proximate to a monitoring device. Operation 504 may be performed by one or more hardware processors configured by machine-readable instructions including a module that is the same as or similar to type class detection module 410, in accordance with one or more implementations.

An operation 506 may include responding to the detection in a manner that ameliorates the harmful effects of the detected type or class of biological cells and/or substances. Operation 506 may be performed by one or more hardware processors configured by machine-readable instructions including a module that is the same as or similar to detection responding module 412, in accordance with one or more implementations.

FIG. 5B illustrates method 500, in accordance with one or more implementations.

An operation 508 may include further including obtaining environmental data based on an environmental factor associated with the in-scene biological cells and/or substances or the environment surrounding the in-scene biological cells and/or substances. Operation 508 may be performed by one or more hardware processors configured by machine-readable instructions including a module that is the same as or similar to data obtaining module 414, in accordance with one or more implementations.

### Glossary

The following is a list of relevant terms used herein. Unless the context in which the term is used indicates differently, the terms of this glossary may be understood as being described in this glossary in accordance with the technology described herein.

Electronic Device: An apparatus that includes one or more electronic components designed to control or regulate the flow of electrical currents for the purpose of information processing or system control. An electronic device may include some mechanical, optical, and/or otherwise non-electronic parts in addition to its electronic components. Examples of such electronic components include transistors, diodes, capacitors, integrated circuits, and the like. Often such devices have one or more processors that are capable of executing instructions, memories, input/output mechanisms (e.g., display screens, touchscreens, cameras, etc.), and communication systems (e.g., wireless networking and cellular telephony). Examples of an electronic device contemplated herein includes a smartphone, a tablet computer, medical equipment, a microscope, a smartdevice, a computer, a standalone unit, a collection of cooperative units, a button-sized unit, system-on-a-chip, a device on a chip, an accessory to a smartphone or smartdevice, an ambulatory device, a robot, swallowability device, an injectable device, or the like. Depending on the implementation, the electronic device may be characterized as: portable; handheld; fits into a typical pocket; lightweight; portable and with fixed (non-aimable) optics—thus, the device must be moved to aim the optics; with aimable optics—thus, the device need not be moved to aim the optics; or a combination thereof. In addition, an implementation of an electronic device may be characterized as a smartdevice (e.g., smartphone or tablet computer) with its own processor and camera (as its scene-capture system); an accessory or case for a smartdevice that operatively attaches to the smartdevice and adds additionally processing capabilities and functionalities for its scene-capture system; stand-alone device with its own processor and camera (as its scene-capture system); ambulatory device that can move under its own power; a device-on-a-chip; system-on-a-chip; or a wireless device that is configured to interconnect with a wireless network of such devices, this device has its own processor camera (as its scene-capture system).

System: An assemblage or combination of things, parts, or components that form a unitary or cooperative whole. In some instances, a system and platform are used synonymously.

Scene: An area, place, location, scenario, etc. that is in view of the scene-capture system.

Image: An array (e.g., two-dimensional) of data derived from and mapped to a scene. An image may be an array of measured data regarding the electromagnetic spectrum emanating from, reflected off, passing through, scattering off of, etc. the contents (e.g., matter) of the scene. The image has an inherent frame or bound around or surrounding the subject scene.

In situ: Describes something that is situated in the original, natural, or existing place or position. Something that is in place or position. It is undisturbed.

In-the-field: A synonym for in situ.

In the lab: Describes the opposite of in situ. That is, it describes something that has been removed from its original or natural place or position. It is something that is not in place. It has been repositioned.

Biological cell: In biology, a cell is the basic structural, functional, and biological unit of all known living organisms. Typically, biological cells consist of cytoplasm enclosed within a membrane, which contains many biomolecules such as proteins and nucleic acids. Organisms can be classified as single-celled or unicellular (consisting of a single cell; including bacteria) or multicellular (such as plants and animals). While the multicellular plants and animals are often visible to the unaided human eye, their individual cells are visible only under a microscope, with dimensions between 1 and 100 micrometers.

Biological substance: As used herein, a biological substance is not itself a biological cell. Rather, this is a substance is strongly associated with biological cells or lifeforms. In particular, a biological substance maybe part of or produced by a biological cell or lifeform. In other instances, a biological substance is capable of affecting a lifeform (or some portion thereof).

Biological cells and/or substances: As used herein, this refers to both “biological cells” and “biological substances.”

Type or class of biological cell: The cells may be classified, categorized, or typed based on identifiable characteristics (e.g., physical, chemical, behavioral, etc.). For example, some cells may be classified as pathological because they cause disease. Some may be a diseased typed because they are malfunctioning and/or infected.

Micrographic: An image is classified as micrographic when it captures content that is on a microscopic scale. Such content includes things that are less than 100 micrometers in size. More generally, it includes items smaller than a macroscopic scale (which are visible to the unaided human eye) and quantum scale (i.e., atomic and subatomic particles).

Spectrographic: An image is classified as spectrographic when it captures the interaction between matter and some portion of the electromagnetic spectrum. Examples of such interactions included absorption, emission, scattering, reflection, refraction, translucency, etc.

Optical: Physics that involves the behavior and properties of light, including its interactions with matter and instruments that use or detect it. However, optics involve more than just the visible spectrum.

Visible Spectrum: This is part of the spectrographic image but specifically includes some portion of the visible spectrum (i.e., light) and excludes the non-visible portions.

Digital: This describes data that is formatted and arranged so as to be managed and stored by a machine, computer, digital electronic device, or the like. A data in the form of a digital signal uses discrete steps to transfer information.

Disease: Any disordered or malfunctioning lifeform or some portion thereof. A diseased lifeform is still alive but ill, sick, ailing, or the like.

Pathological: Something that is capable of causing disease or malfunction in a lifeform (or a portion thereof). A pathogen is pathological.

Pathobiological: Something is pathobiological if it is either capable of causing the disease to a lifeform (or some portion thereof) or is a diseased lifeform (or some portion thereof).

Pathobiological cell: A biological cell that is pathobiological.

Pathobiological substance: This is a substance that is either capable of causing the disease to a lifeform (or some portion thereof) or is associated with a diseased lifeform (or some portion thereof). The substance is not itself a biological cell.

Pathobiological cells and/or substances: As used herein, the term “pathobiological” modifies both “cell” and “substance.”

Pathogen: A biological cell (e.g., unicellular organism) that is capable of causing a disease. More generally, anything that can cause or produce disease.

Diseased cell: A biological cell (e.g., cancerous cell) that is alive but diseased.

Lifeform: The body form that characterizes an organism. Examples of lifeforms include:
Plants—Multicellular, photosynthetic eukaryotes
Animals—Multicellular, eukaryotic organisms
Fungus—Eukaryotic organisms that include microorganisms such as yeasts and molds
Protists—a Eukaryotic organism that is not an animal, plant or fungus
Archaea—Single-celled microorganisms
Bacteria—Prokaryotic microorganisms

Organism: An organism may generally be characterized as containing different levels of the organization; utilizing energy; responding to stimuli/environment; maintaining homeostasis; undergoing metabolism; growing; reproduction; and adapting to its environment.

Environmental factor: It is anything measurable that is capable of affecting the scene or that is associated with the scene. Such things can be abiotic or biotic. Abiotic factors include, for example, ambient temperature, moisture, humidity, radiation, the amount of sunlight, and pH of the water medium (e.g., soil) where a microbe lives. Examples of biotic factors include the availability of food organisms and the presence of conspecifics, competitors, predators, and parasites.

Smartphone: Generally, this term refers to a portable electronic device with features that are useful for mobile communication and computing usage. Such features the ability to place and receive voice/video calls, create and receive text messages, an event calendar, a media player, video games, GPS navigation, digital camera and video camera.

Smartdevice: The concept of a smartdevice includes a smartphone, but it also includes any other portable electronic device that might not have all of the features and functionality of a smartphone. Examples of a smartdevice include a tablet computer, portable digital assistant, smart watches, fitness tracker, location trackers, a so-called internet-of-things device, and the like. A smartdevice is an electronic device that is generally connected to other devices or networks via different wireless protocols such as Bluetooth, NFC, Wi-Fi, 3G, etc., that can operate to some extent interactively and autonomously.

Accessory: As used herein, this is an accessory to an electronic device (such as a smartphone or smartdevice). It adds additional functionality and/or capabilities to the electronic device. Examples of such accessories include a smartwatch or electronically enabled phone case.

Therapeutic: A treatment intended to have a curative, medicinal, preventative, remedial, or otherwise beneficial effect on the bodies or minds of one or more persons. Such therapeutics may, for example, directly or indirectly treat diseases or disorders. Such therapeutics may, for example, promote wellness and/or discourage disease or disorders. Such therapeutics may, for example, address symptoms of diseases or disorders. Examples of therapeutics includes medicines, drugs, procedures, medical treatments, medical therapies, stress reduction therapies, and the like.

### Other Applications

In addition to the example scenarios and applications discussed above, the following are other example scenarios and applications in which the technology described herein may be employed. Of course, there are still other scenarios and applications in which the technology described herein may be employed, but they are not listed here.

Hospital cleanliness: Using a handheld device, the surfaces and equipment may be regularly checked to confirm their cleanliest and alert for the need to redouble sanitation/cleanliness procedures. Robot or human cleaners may be dispatched to clean or sanitize flagged areas and equipment. Other forms of a device (e.g., robot, mounted device, etc.) may be used for the same purposes.

In-room monitoring: Using one or more of small wireless communicating devices, critical areas may be continuously monitored for any dangers (e.g., pathogens). For example, a fixed device may be placed in the HVAC ducting of a building to monitor the presence of potentially harmful bacteria or allergens in the ventilation system. In another example, an ambulatory device (e.g., robot) may travel around a room looking for potentially infectious agents. When trouble spots are found, then it may be flagged for priority on the existing cleaning and sanitization schedule and/or robots or humans may be dispatched to clean and sanitize the flagged troubled spots.

For example, rooms may be actively monitored for air quality. Devices may actively monitor for extreme and subtle room level exposure to dangerous VOCs, particulate matter through highly sensitive on-board sensor technology. In addition, devices may monitor for conditions conducive to the growth and spread of pathogens through temperature and humidity sensors

Application of sanitizers and cleaners: A robotic version of the electronic device may be particularly suited for both detecting potentially dangerous pathogens and neutralizing the threat by delivering sanitizing and/or cleaning agents to an area inhabited by the dangerous pathogens.

On-person monitoring: Using a device-on-a-chip approach, a person may discretely wear a device designed to monitor the surfaces and liquids that the person encounters each day. Alternatively, the device may be attached to the person herself to monitor her skin or anything on the skin.

For example, a person may wear a personal air quality monitoring device that actively monitors the air quality of the air that the person is breathing. Such device may actively monitor for extreme and subtle personal exposure to dangerous VOCs, particulate matter through highly sensitive on-board sensor technology. In addition, such devices may monitor for conditions conducive to the growth and spread of pathogens through temperature and humidity sensors.

For example, a person may wear a passive wearable molecular and cellular collection device. Such device may passively collect and preserve a sampling of an individual's chemical and pathogen exposure, for example, daily. Such device will contain a set (e.g., 30) of collection cartridges that are capable of retaining samples of ambient material to which the person is exposed. For example, samples of the biological cells and/or substances proximate to or surrounding that person may be collected. The collected samples are later analyzed.

The approximate time of the samples collected is linked to the timestamp of when the collection cartridge was actively collecting. For example, One cartridge may be exposed per day for sample collection and rotated into preservation mode at the end of the day, exposing a new cartridge for the next day of collection. At the end of the month, the wearable can be removed and mailed in for thorough chemical and biological lab-based analysis to create 30-day exposure overview for the individual.

For example, a person may wear a personal molecular monitor. Such device may actively monitor for extreme and subtle personal exposure to dangerous VOCs, particulate matter through highly sensitive on-board sensor technology. In addition, such devices Actively samples and monitors selection of biomarker indicators of health through transdermal collection mechanism.

In vivo monitoring and delivery: A miniaturized device may be injected into the bloodstream of an animal or human. The device may passively flow with the blood, or it may have its own propulsion system. This device seeks out diseased cells (e.g., cancer) in the bloodstream or in tissues accessible therefrom. Alternatively, the device is implanted into tissue or ingested. From there, it may release therapeutics when appropriate conditions arise.

Application of therapeutic: A version of the device may be placed on or in a living body as a wearable or implantable. This device may respond to the detection of disease, disorder, or indicators of such by delivering therapeutics designed to treat that disease, ease symptoms, or otherwise benefit the person.

For example, a person may wear a personal molecular monitor and therapeutic delivery device. Such device may actively monitor for extreme and subtle personal exposure to dangerous VOCs, particular matter and conditions conducive to the growth and spread of pathogens. This device actively samples and monitors selection of biomarker indicators of health through transdermal collection mechanism. The device actively transdermally releases preloaded therapeutic (e.g., drug) based on exposure and resulting biomarkers specific to healthy individual or the management of a given disease state.

### ADDITIONAL AND ALTERNATIVE IMPLEMENTATION NOTES

In the above description of example implementations, for purposes of explanation, specific numbers, materials configurations, and other details are set forth in order to better explain the present disclosure. However, it will be apparent to one skilled in the art that the subject matter of the claims may be practiced using different details than the examples ones described herein. In other instances, well-known features are omitted or simplified to clarify the description of the example implementations.

The terms “techniques” or “technologies” may refer to one or more devices, apparatuses, systems, methods, articles of manufacture, and/or executable instructions as indicated by the context described herein.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

These processes are illustrated as a collection of blocks in a logical flow graph, which represents a sequence of operations that may be implemented in mechanics alone, with hardware, and/or with hardware in combination with firmware or software. In the context of software/firmware, the blocks represent instructions stored on one or more non-transitory computer-readable storage media that, when executed by one or more processors or controllers, perform the recited operations.

Note that the order in which the processes are described is not intended to be construed as a limitation, and any number of the described process blocks can be combined in any order to implement the processes or an alternate process. Additionally, individual blocks may be deleted from the processes without departing from the spirit and scope of the subject matter described herein.

The term “computer-readable media” is non-transitory computer-storage media or non-transitory computer-readable storage media. For example, computer-storage media or computer-readable storage media may include, but are not limited to, magnetic storage devices (e.g., hard disk, floppy disk, and magnetic strips), optical disks (e.g., compact disk (CD) and digital versatile disk (DVD)), smart cards, flash memory devices (e.g., thumb drive, stick, key drive, and SD cards), and volatile and non-volatile memory (e.g., random access memory (RAM), read-only memory (ROM)).

1. A system configured that facilitates amelioration based on detection of biological cells or biological substances, the system comprising:
one or more hardware processors configured by machine-readable instructions to:
monitor a scene proximate to a monitoring device, the scene including biological cells and/or substances;
detect a type or class of biological cells and/or substance amongst the in-scene biological cells and/or substances found in a scene proximate to a monitoring device; and
respond to the detection in a manner that ameliorates the harmful effects of the detected type or class of biological cells and/or substances.

2. The system of claim 1, wherein the amelioration action includes introducing an active material to a physical location of the biological cell and/or substance to neutralize the biological nature of the detected type of biological cells and/or substances.

3. The system of claim 1, wherein the amelioration action includes formulating one or more therapeutics for one or more individuals associated with the scene, the formulated therapeutics being configured to treat one or more conditions associated with the detected type of biological cells and/or substances.

4. The system of claim 1, wherein the amelioration action includes selecting one or more therapeutics for one or more individuals associated with the scene, the selected therapeutics being configured to treat one or more conditions associated with the detected type of biological cells and/or substances.

5. The system of claim 1, wherein the amelioration action includes selecting one or more therapeutics for one or more individuals associated with the scene and a dosage of such therapeutics, the selected dosage of the therapeutics being configured to treat one or more conditions associated with the detected type of biological cells and/or substances.

6. The system of claim 1, wherein the amelioration action includes introducing therapeutic to a physical location of the biological cell and/or substance to neutralize the biological nature of the detected type of biological cells and/or substances.

7. The system of claim 1, wherein the amelioration action includes introducing therapeutics to one or more individuals associated with the scene, the therapeutics being configured to treat one or more conditions associated with the detected type of biological cells and/or substances.

8. The system of claim 7, wherein the therapeutics is introduced to the one or more individuals before the one or more individuals have presented any or sufficient symptoms to otherwise warrant the therapeutics to treat one or more conditions associated with the detected type of biological cells and/or substances.

9. The system of claim 1, wherein the amelioration action includes dispatching or requesting a visit by a robot or human to a physical location of the biological cell and/or substance to neutralize the biological nature of the detected type of biological cells and/or substances.

10. The system of claim 1, wherein the amelioration action includes dispatching or requesting a visit by a robot or human to a physical location of the detected type of biological cells and/or substances to document the conditions around the detected type of biological cells and/or substances.

11. The system of claim 1, wherein the amelioration action includes activating an operation of a proximate electronic device or system that is proximate a physical location of the detected type of biological cells and/or substances to neutralize the biological nature of the detected type of biological cells and/or substances.

12. The system of claim 1, wherein the amelioration action includes activating an operation of a proximate camera that is proximate a physical location of the detected type of biological cells and/or substances to document the area around the detected type of biological cells and/or substances.

13. The system of claim 1, wherein the amelioration action includes internal releasing preloaded therapeutic to one or more individuals associated with the scene by one or more implanted therapeutic delivery devices.

14. The system of claim 1, wherein the amelioration action includes transdermally releasing preloaded therapeutic to one or more individuals associated with the scene by one or more transdermal therapeutic delivery devices.

15. The system of claim 1, wherein the amelioration action includes topical application of a dosage of a therapeutic to from one or more individuals associated with the scene by one or more dermal therapeutic delivery devices.

16. The system of claim 1, wherein the amelioration action includes aeriated release of an airborne therapeutic in an area that includes or is proximate to the scene being monitored by one or more therapeutic delivery devices.

17. The system of claim 1, wherein the amelioration action includes physical delivery of a package containing a therapeutic for one or more individuals associated with the scene.

18. The system of claim 1, wherein the monitoring and detecting actions are performed physically and temporally separate and at a distance from each other.

19. The system of claim 1, wherein the monitoring includes retaining samples of the in-scene biological cells and/or substances during a period of time for subsequent analysis.

20. The system of claim 1, wherein the monitoring includes actively monitoring a selection of biomarker indicators of health through transdermal collection.

21-152. (canceled)
